(19) United States
(12) Patent Application Publication
(10) Pub. No.: US 2025/0339302 A1
(43) Pub. Date: Nov. 6, 2025

(54) ORTHOPEDIC LIMB BRACE AND METHOD THEREFOR

(71) Applicants: Juliette Steffensen, Greensburg, PA (US); Walker Watson, Fairhope, AL (US); Ryan Poquis, Glen Allen, VA (US); Colin Berry, Charlottesville, VA (US); Thomas Chia, Centreville, VA (US)

(72) Inventors: Juliette Steffensen, Greensburg, PA (US); Walker Watson, Fairhope, AL (US); Ryan Poquis, Glen Allen, VA (US); Colin Berry, Charlottesville, VA (US); Thomas Chia, Centreville, VA (US)

(21) Appl. No.: 18/653,481

(22) Filed: May 2, 2024

(51) Int. Cl. A61F 5/01

(52) U.S. Cl. CPC A61F 5/013; A61F 2005/0155; A61F 2005/0158; A61F 2005/0172

(57) ABSTRACT

An orthopedic limb brace includes a brace frame, a pair of shells, a pair of securing cuff assemblies, and a pair of tightening assemblies. The brace frame includes a pair of arm structures pivotably connected together by an adjustable ROM joint. Each securing cuff assembly is detachably connected to a corresponding one of the shells, wherein the securing cuff assemblies are designed to be wrapped around and detachably secured to portions of a limb of a wearer, one each on either side of a limb joint. Each tightening assembly includes a flexible cord attached to a corresponding one of the securing cuff assemblies, a winding mechanism connected to and designed to wind and unwind the cord to tighten and release its corresponding securing cuff assembly, and a base structure mounted to a corresponding shell and designed to detachably secure the winding mechanism to its corresponding shell by magnetic force.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to orthopedic braces and rehabilitation accessories and more particularly pertains to a new orthopedic limb brace that is easily secured and tightened to the limb of a user, such as a user's arm, with minimal effort and using one hand, and which exhibits very little or minimal migration along the limb during use. Current orthopedic limb braces are bulky and have complex attachment mechanisms that cause the wearer to exert considerable effort to attach the brace to a limb, especially an arm since the wearer has only one free hand to use, which discourages use and proper fit and thereby inhibits recovery. In addition, current orthopedic limb braces exhibit substantial and undesirable migration in that they unintentionally slide or move along the limb during use due to the bulky size, difficulty in properly securing the brace, and inadequate design.

### (2) DESCRIPTION OF RELATED ART INCLUDING INFORMATION DISCLOSED UNDER 37 CFR 1.97 AND 1.98

The prior art relates to orthopedic braces and rehabilitation accessories. The prior art, as best understood, does not disclose an orthopedic limb brace that is easily secured and tightened to the limb of a user, such as a user's arm, with minimal effort and using one hand, and which exhibits very little or minimal migration along the limb during use. The orthopedic limb brace includes a brace frame, a pair of shells, a pair of securing cuff assemblies, and a pair of tightening assemblies, wherein each of the pair of securing cuff assemblies is detachably connected to a corresponding one of the shells, and each of the pair of tightening assemblies has a winding mechanism and a base structure that secures the winding mechanism to its corresponding shell by magnetic force.

### (h) BRIEF SUMMARY OF THE INVENTION

An embodiment of the disclosure meets the needs presented above in an orthopedic limb brace generally comprising a brace frame, a pair of shells, a pair of securing cuff assemblies, and a pair of tightening assemblies. The brace frame includes a pair of arm structures pivotably connected together by an adjustable range of motion joint. Each of the pair of shells is curved and slidably mounted on a corresponding one of the arm structures. Each of the pair of securing cuff assemblies is detachably connected to a corresponding one of the shells, wherein the securing cuff assemblies are designed to be wrapped around and detachably secured to portions of a limb of a wearer, one each on either side of a limb joint. Each of the pair of tightening assemblies includes a flexible cord attached to a corresponding one of the securing cuff assemblies, a winding mechanism connected to and designed to wind and unwind the cord to tighten and release its corresponding securing cuff assembly, and a base structure mounted to a corresponding one of the shells and designed to detachably secure the winding mechanism to its corresponding shell by magnetic force. In addition, each of the securing cuff assemblies includes a friction structure positioned at an inner surface thereof that is designed to minimize unintentional sliding movement of the orthopedic limb brace along a limb of a wearer.

The orthopedic limb brace can first be partially placed about an arm of a wearer. The wearer can then manually secure and tighten each of the tightening assemblies with only one hand. The wearer can then release each of the tightening assemblies with only one hand.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of an orthopedic limb brace according to an embodiment of the disclosure.

FIG. 2 is a front perspective view of an embodiment of the disclosure partially disassembled.

FIG. 3 is a rear perspective view of an embodiment of the disclosure.

FIG. 4 is a close-up view of a portion of an embodiment of the disclosure.

FIG. 5 is a top view of a securing cuff assembly of an embodiment of the disclosure.

FIG. 6 is a cross-sectional view of a securing cuff assembly of an embodiment of the disclosure.

FIG. 7 is an exploded view of a component of an embodiment of the disclosure.

FIG. 8 is an exploded view of a component of an embodiment of the disclosure.

FIG. 9 is a side view of an embodiment of the disclosure in use.

FIG. 10 is a side view of a portion of an embodiment of the disclosure in use.

### (j) DETAILED DESCRIPTION OF THE INVENTION

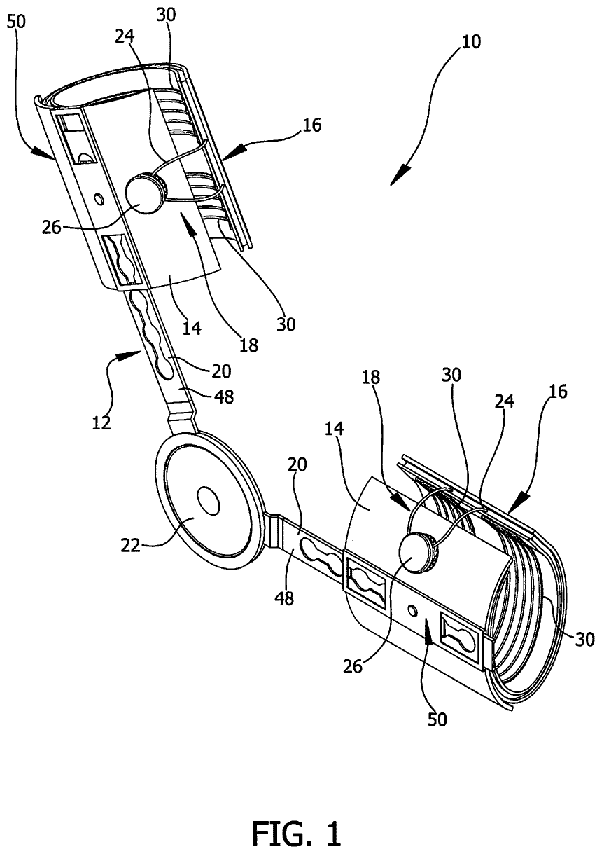

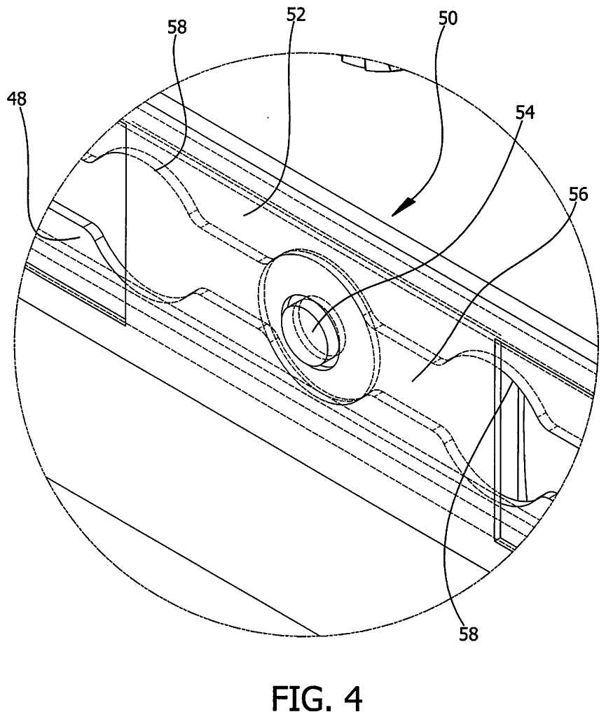

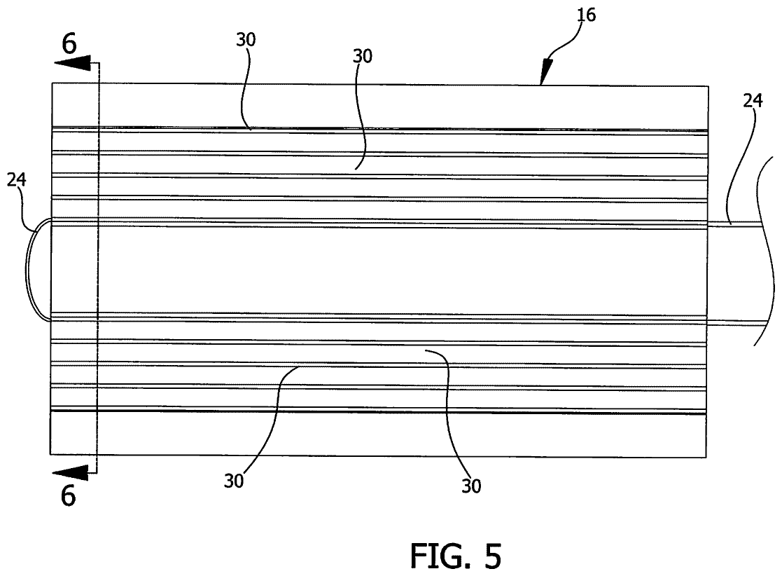

With reference now to the drawings, and in particular to FIGS. 1 through 10 thereof, a new orthopedic limb brace embodying the principles and concepts of an embodiment of the disclosure and generally designated by the reference numeral 10 will be described.

As best illustrated in FIGS. 1 through 10, the orthopedic limb brace 10 generally comprises a brace frame 12, a pair of shells 14, a pair of securing cuff assemblies 16, and a pair of tightening assemblies 18. The brace frame 12 includes a pair of arm structures pivotably connected together by an adjustable range of motion joint 22. Each of the pair of shells 14 is curved and slidably mounted on a corresponding one of the arm structures 20. Each of the pair of securing cuff assemblies 16 is detachably connected to a corresponding one of the shells 14, wherein the securing cuff assemblies 16 are designed to be wrapped around and detachably secured to portions of a limb of a wearer, one each on either side of a limb joint. Each of the pair of tightening assemblies 18 includes a flexible cord 24 attached to a corresponding one of the securing cuff assemblies 16, a winding mechanism 26 connected to and designed to wind and unwind the cord 24 to tighten and release its corresponding securing cuff assembly 16, and a base structure 28 mounted to a corresponding one of the shells 14 and designed to detachably secure the winding mechanism 26 to its corresponding shell 14 by magnetic force.

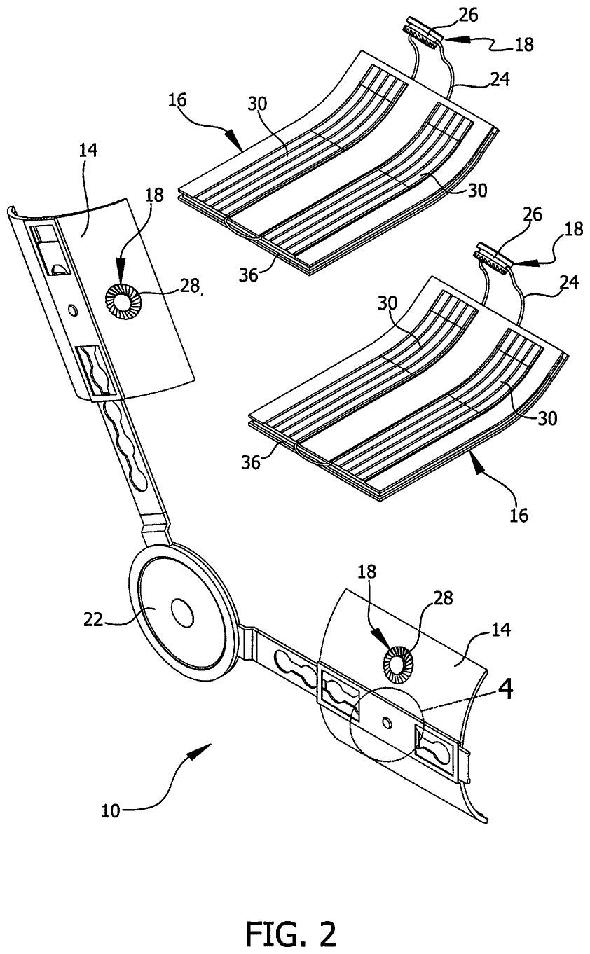

As seen in FIGS. 2, 5, and 6, each of the securing cuff assemblies 16 includes a friction structure 30 positioned at an inner surface thereof and designed to minimize unintentional sliding movement of the orthopedic limb brace 10 along a limb of a wearer. In the exemplary embodiment shown, the friction structure 30 can be a soft hypoallergenic material, such as silicone or a similar medical-grade material that grips against the skin without significant or noticeable irritation. The friction structure 30 can be in the form of a plurality of raised elongated strips, such as nylon cords encased or covered by silicone, though other shapes and designs that provide sufficient friction to minimize migration without harming the wearer's skin are within the scope of the disclosure.

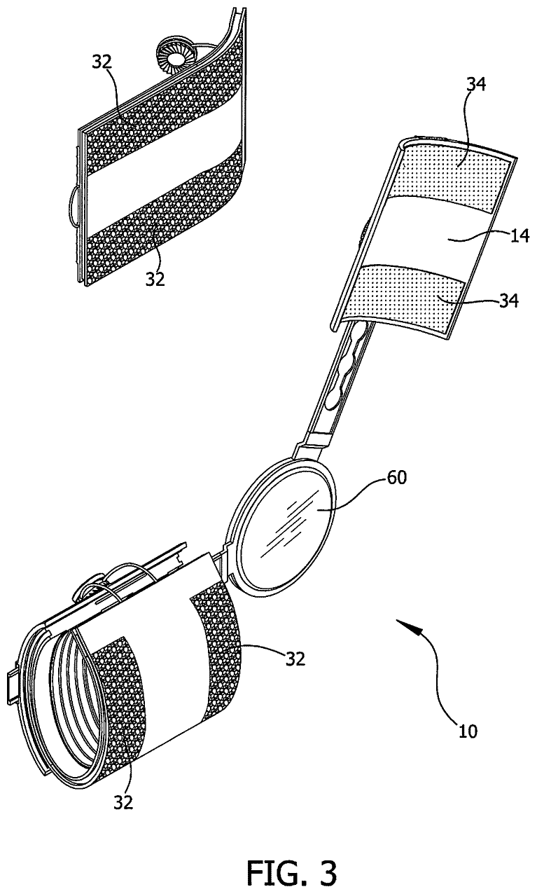

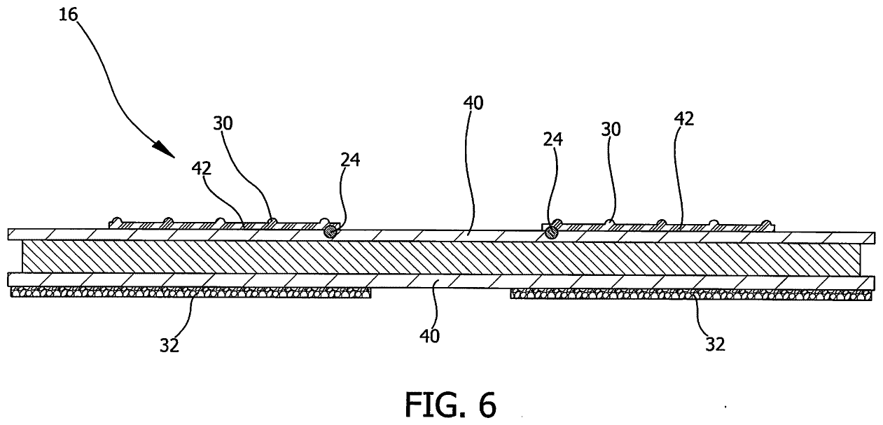

As seen in FIGS. 3 and 6, each of the securing cuff assemblies 16 includes a loop material 32 attached to an outer surface thereof and each of the shells 14 includes a hook material 34 attached to an interior surface thereof to permit detachable connection and selective positioning of the securing cuff assemblies 16 on the shells 14. In the exemplary embodiment, there are two strips of loop material 32 and two strips of hook material 34. Other detachable connection structures, such as fasteners or clips, are within the scope of the disclosure. Each of the securing cuff assemblies 16 has a layered structure 36 that includes layers of fabric material and cushioning material. Depending on the desired properties, at least one of the layers includes a waterproof material, or an absorbent or moisture-wicking material, or a resiliently stretchable material, or a material with a combination of one or more of those characteristics. As shown in FIGS. 2 and 6, each of the flexible cords 24 is in the form of a closed loop of resilient elastic material positioned to run lengthwise through and project out of a corresponding one of the securing cuff assemblies 16. FIG. 6 shows a possible design of the securing cuff assembly 16, wherein there is a center layer 38 of cushioning material, such as neoprene or scuba material, sandwiched between two fabric layers 40, such as gaberdine material or other fabric. Two strips of loop material 32 are located on the underside. Two strips of absorbent or sweat-wicking elastic material 42 are located on the opposite side and have friction structures 30 located on the top thereof, which could be nylon cords encased or covered by silicone. Other fabric or cloth materials made of natural or artificial fibers, or elastomers or foam or rubber materials, or metallic or plastic materials and components could be utilized in different layers, widths, lengths, and thicknesses. The securing cuff assemblies 16 can be made of washable materials to permit a user to detach and clean the securing cuff assemblies 16 of dirt and sweat.

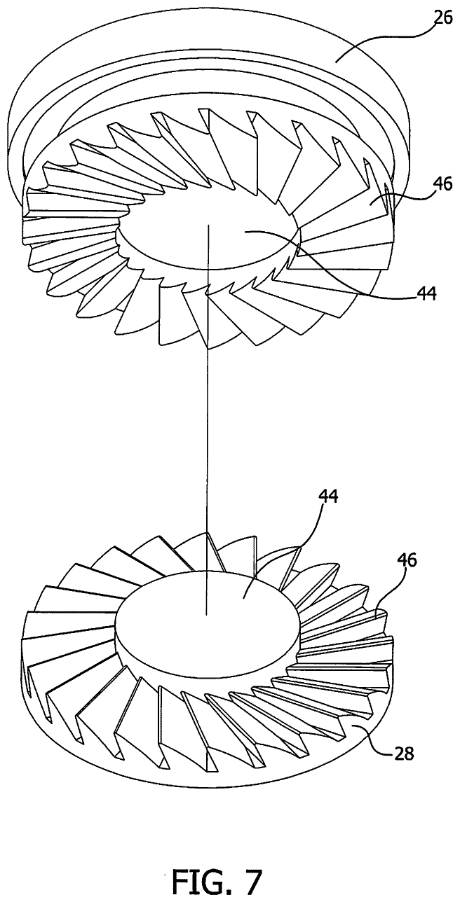

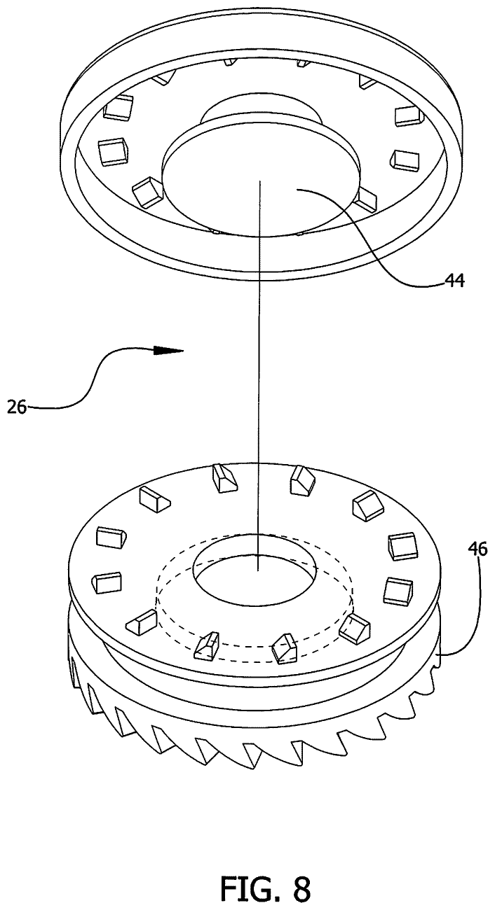

With reference to FIGS. 7 and 8, each of the winding mechanism 26 and the base structure 28 includes a central piece 44 of magnetic material having a flat end face that permits rotation of the winding mechanism 26 with respect to the base structure 28. Each of the winding mechanism 26 and the base structure 28 also includes a plurality of angled mating teeth 46 to permit rotation and selective locking of the winding mechanism 26.

As shown in FIGS. 1, 2, and 4, each of the arm structures 20 includes a strip 48 of metal or hard plastic and each of the shells 14 includes a releasable locking assembly 50 designed to receive and selectively lock its corresponding shell 14 in a desired position on the strip 48. The releasable locking assembly 50 includes a mounting passage 52 through which the strip 48 is removably inserted and a push button 54 projecting out through an outer wall of the mounting passage 52, wherein each of the strips 48 includes a contoured slot 56 having locking openings 58 designed to receive the push button 54 to permit selective positioning of its corresponding shell 14 on the strip 48. Each of the shells 14 includes a resiliently flexible plastic material, which allows for a tighter and more secure connection as the shells 14 can be bent or curved to conform to a limb. Each of the shells 14 has a width about half of the length of the securing cuff assembly 16 and a low cross-sectional profile of less than about 0.5 inch. The total overall thickness of the orthopedic limb brace 10, including the shells 14 and the securing cuff assemblies 16, is an inch or less. This low-profile shape provides a very lightweight design and comfortable user experience. In addition, the range of motion (ROM) joint 22 includes a layer of padding material 60 on an interior surface thereof.

In testing, the inventors found that migration of the orthopedic limb brace 10 along an arm was reduced to about 0.5 inch or less when worn during a 10-minute walk. No migration occurred during a 10-second shake test. Installation of the orthopedic limb brace 10 could be accomplished very quickly using one hand, usually within 60 seconds or less. Connection of all attachment points could be performed in less than 15 seconds with one hand. Removal could be performed in less than 30 seconds with one hand.

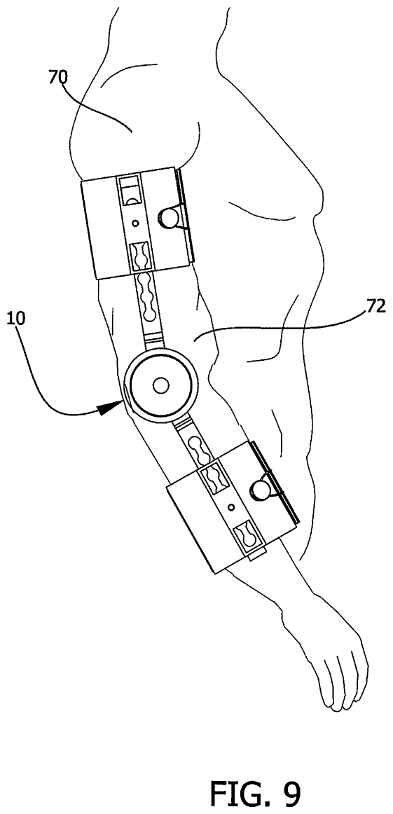

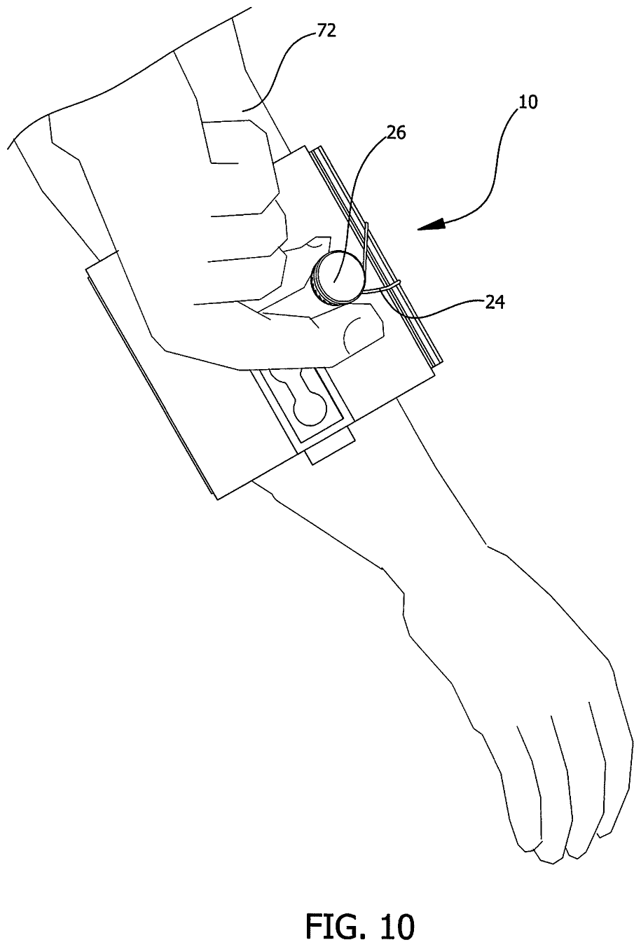

The orthopedic limb brace 10 is used to brace a limb for rehabilitation. As shown, for example, in FIGS. 9 and 10, the wearer 70 first places the orthopedic limb brace 10 partially about his or her arm 72. The securing cuff assemblies 16 are pulled around the arm 72 and the winding mechanism 26 is magnetically connected to the base structure 28. At this point the orthopedic limb brace 10 is loosely attached to the wearer's arm 72. As shown in FIG. 10, the wearer 70 then adjusts the winding mechanism 26 by rotation, which winds up the flexible cord 24 and tightens up the securing cuff assembly 16, thereby securing the orthopedic limb brace 10 to the wearer's arm 72. The winding mechanism 26 functions like a ratchet or Fidlock® winch due to the angled mating teeth 46, which allow the winding mechanism 26 to rotate in one direction, but lock and prevent rotation in the opposite direction. The steps of manually securing and tightening each of the tightening assemblies 18 can be performed by the wearer 70 with only one hand. After use or to readjust the position of the orthopedic limb brace 10, the wearer 70 can release each of the tightening assemblies 18 with only one hand by simply pulling off the winding mechanism 26 and unwinding the flexible cord 24. The steps of placing, securing, and tightening can be completed very quickly, such as under 60 seconds as discussed above.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An orthopedic limb brace comprising:
a brace frame comprising a pair of arm structures pivotably connected together by an adjustable range of motion joint;
a pair of shells, each being curved and slidably mounted on a corresponding one of said arm structures;
a pair of securing cuff assemblies, each being detachably connected to a corresponding one of said shells, wherein said securing cuff assemblies are configured to be wrapped around and detachably secured to portions of a limb of a wearer, one each on either side of a limb joint; and
a pair of tightening assemblies, each comprising a flexible cord attached to a corresponding one of said securing cuff assemblies, a winding mechanism connected to and configured to wind and unwind said cord to tighten and release its corresponding securing cuff assembly, and a base structure mounted to a corresponding one of said shells and configured to detachably secure said winding mechanism to its corresponding shell by magnetic force.

2. The orthopedic limb brace of claim 1, wherein each of said securing cuff assemblies comprises a friction structure disposed at an inner surface thereof and configured to minimize unintentional sliding movement of the orthopedic limb brace along a limb of a wearer.

3. The orthopedic limb brace of claim 2, wherein said friction structure comprises a soft hypoallergenic material.

4. The orthopedic limb brace of claim 2, wherein said friction structure comprises a plurality of raised elongated strips.

5. The orthopedic limb brace of claim 2, wherein each of said securing cuff assemblies comprises a loop material attached to an outer surface thereof and each of said shells comprises a hook material attached to an interior surface thereof to permit detachable connection and selective positioning of said securing cuff assemblies on said shells.

6. The orthopedic limb brace of claim 2, wherein each of said securing cuff assemblies has a layered structure comprising layers of fabric material and cushioning material, each of which comprises a washable material.

7. The orthopedic limb brace of claim 6, wherein at least one of said layers comprises a waterproof material.

8. The orthopedic limb brace of claim 6, wherein at least one of said layers comprises an absorbent material.

9. The orthopedic limb brace of claim 6, wherein at least one of said layers comprises a resiliently stretchable material.

10. The orthopedic limb brace of claim 2, wherein each of said flexible cords is comprises a closed loop of resilient elastic material disposed to run lengthwise through and project out of a corresponding one of said securing cuff assemblies.

11. The orthopedic limb brace of claim 2, wherein each of said winding mechanism and said base structure comprises a central piece of magnetic material having a flat end face that permits rotation of said winding mechanism with respect to said base structure.

12. The orthopedic limb brace of claim 2, wherein each of said winding mechanism and said base structure comprises a plurality of angled mating teeth to permit rotation and selective locking of said winding mechanism.

13. The orthopedic limb brace of claim 2, wherein each of said arm structures comprises a strip of metal or hard plastic and each of said shells comprises a releasable locking assembly configured to receive and selectively lock its corresponding shell in a desired position on said strip.

14. The orthopedic limb brace of claim 13, wherein said releasable locking assembly comprises a mounting passage through which said strip is removably inserted and a push button projecting out through an outer wall of said mounting passage, wherein each of said strips comprises a contoured slot having locking openings configured to receive said push button to permit selective positioning of its corresponding shell on said strip.

15. The orthopedic limb brace of claim 2, wherein each of said shells comprises a resiliently flexible plastic material.

16. The orthopedic limb brace of claim 2, wherein each of said shells has a width about half of the length of said securing cuff assembly and a low cross-sectional profile of less than about 0.5 inch.

17. The orthopedic limb brace of claim 2, wherein said range of motion joint comprises a layer of padding material on an interior surface thereof.

18. A method of using the orthopedic limb brace of claim 2, comprising the steps of:
placing the orthopedic limb brace partially about an arm of a wearer; and
manually securing and tightening each of said tightening assemblies with only one hand of a wearer; and
releasing each of said tightening assemblies with only one hand of a wearer.

19. The method of claim 18, wherein said steps of placing, securing, and tightening are completed in under 60 seconds.

20. An orthopedic limb brace comprising:
a brace frame comprising a pair of arm structures pivotably connected together by an adjustable range of motion joint, wherein:
said range of motion joint comprises a layer of padding material on an interior surface thereof;
a pair of shells, each being curved and slidably mounted on a corresponding one of said arm structures, wherein:
each of said arm structures comprises a strip of metal or hard plastic and each of said shells comprises a releasable locking assembly configured to receive and selectively lock its corresponding shell in a desired position on said strip, wherein:
said releasable locking assembly comprises a mounting passage through which said strip is removably inserted and a push button projecting out through an outer wall of said mounting passage, wherein each of said strips comprises a contoured slot having locking openings configured to receive said push button to permit selective positioning of its corresponding shell on said strip,
each of said shells comprises a resiliently flexible plastic material,
each of said shells has a width about half of the length of said securing cuff assembly,
each of said shells has a low cross-sectional profile of less than about 0.5 inch;
a pair of securing cuff assemblies, each being detachably connected to a corresponding one of said shells, wherein said securing cuff assemblies are configured to be wrapped around and detachably secured to portions of a limb of a wearer, one each on either side of a limb joint, wherein:
each of said securing cuff assemblies comprises a friction structure disposed at an inner surface thereof and configured to minimize unintentional sliding movement of the orthopedic limb brace along a limb of a wearer,
said friction structure comprises a soft hypoallergenic material, said friction structure comprises a plurality of raised elongated strips,
each of said securing cuff assemblies comprises a loop material attached to an outer surface thereof and each of said shells comprises a hook material attached to an interior surface thereof to permit detachable connection and selective positioning of said securing cuff assemblies on said shells,
each of said securing cuff assemblies has a layered structure comprising layers of fabric material and cushioning material, wherein at least one of:
at least one of said layers comprises a waterproof material,
at least one of said layers comprises an absorbent material,
at least one of said layers comprises a resiliently stretchable material,
each of said layers comprises a washable material; and
a pair of tightening assemblies, each comprising a flexible cord attached to a corresponding one of said securing cuff assemblies, a winding mechanism connected to and configured to wind and unwind said cord to tighten and release its corresponding securing cuff assembly, and a base structure mounted to a corresponding one of said shells and configured to detachably secure said winding mechanism to its corresponding shell by magnetic force, wherein:
each of said flexible cords is disposed to run lengthwise through and project out of a corresponding one of said securing cuff assemblies,
each of said flexible cords comprises a closed loop of resilient elastic material,
each of said winding mechanism and said base structure comprises a central piece of magnetic material having a flat end face that permits rotation of said winding mechanism with respect to said base structure,
each of said winding mechanism and said base structure comprises a plurality of angled mating teeth to permit rotation and selective locking of said winding mechanism.
